(19) United States
(12) Patent Application Publication
(10) Pub. No.: US 2016/0166809 A1
(43) Pub. Date: Jun. 16, 2016

(54) Tube System for Medical Applications

(71) Applicant: Raumedic AG, Munchberg (DE)

(72) Inventors: Björn Gläsel, Kirchenlamitz (DE); Axel Wunderlich, Ebmath (DE)

(21) Appl. No.: 14/968,072

(22) Filed: Dec. 14, 2015

(30) Foreign Application Priority Data
Dec. 15, 2014 (DE) 10 2014 225 939.8

(51) Int. Cl. A61M 25/09; A61M 25/00

(52) U.S. Cl. CPC A61M 25/09; A61M 25/0026; A61M 2025/09133; A61M 25/09041; A61M 2205/0238

(57) ABSTRACT

A tube system for medical application has a tube with at least one tube lumen. A guide wire is inserted in at least a tube section in at least one tube lumen. An adhesive coating is present between an outer wall of the inserted guide wire and an inner wall of the guide wire-tube lumen. This results in a tube system with better handling characteristics.

### CROSS-REFERENCES TO RELATED APPLICATIONS

This application claims the priority of German Patent Application, Serial no. 10 2014 225 939.8, filed Dec. 15, 2014, pursuant to 35 U.S.C. 119(a)-(d), the content of which is incorporated herein by reference in its entirety as if fully set forth herein.

### FIELD OF THE INVENTION

The invention relates to a tube system for medical applications.

### BACKGROUND OF THE INVENTION

A tube system of this type is known from DE 31 07 392 A1 and US 2003/0135156 A1.

### SUMMARY OF THE INVENTION

The object of the present invention is to improve the handling of such a tube system.

The object is achieved according to the invention by means of a tube system having the features listed in claim 1.

It has been determined in accordance with the invention that the adhesive coating between the outer wall of the inserted guide wire and the inner wall of the guide wire-tube lumen provides for a fixing of the guide wire in the guide wire-tube lumen and thus for a securing of the guide wire against torsion to in relation to the tube. This improves the handling of the tube system, because, particularly when exerting a torsional force, the tube does not deviate beyond a relative torsion of a torsional force in relation to the internal guide wire in an undesired manner. A cross section profiling of the guide wire, or the guide wire-tube lumen for ensuring a securing against torsion is then not necessary. Accordingly, the guide wire may have a round outer cross section and the guide wire tube lumen may have a round inner cross section. Alternatively, for a supplementary securing against torsion, a corresponding, non-rotationally symmetrical profiling of the outer wall of the guide wire, or the inner wall of the guide wire-tube lumen is possible. The guide wire provides for a stiffening of the tube within the tube system, and thus for an improvement in the guidance. The guide wire may be deformable in an elastic and/or plastic manner.

An adhesive coating according to claim 2 may also cover the guide wire, in particular at the end surfaces. The coating may also be present in the region of the end surfaces of the guide wire. A tight sealing of the guide wire to the outside is possible by this means. This results, in particular, in a protection against corrosion of the guide wire.

A copper wire according to claim 3 may be implemented with silver plating, in particular. Such a design for the guide wire has been proven in the field.

A controlled hardening adhesive according to claim 4 provides for defined conditions when generating the adhesive coating. Prior to hardening, the adhesive may be available as a low viscosity fluid. The adhesive can be a UV-curing adhesive, e.g. having a cyanoacrylate base. Alternatively or additionally it can be an adhesive that hardens under the effects of humidity and/or temperature. An adhesive having a longer curing time is also possible for the formation of the adhesive coating.

A multi-lumen tube according to claim 5 increases the application possibilities for the tube system. The guide wire can be inserted in precisely one lumen, or in numerous lumina of a multi-lumen tube. In particular, an addition of a media or the taking of a sample via the tube of the tube system is made possible.

A three-lumen tube according to claim 6 may be used, in particular, as a dosing system for a two-component fibrin glue.

A four-lumen tube according to claim 7 may be used, in particular, as a dosing system for applying a protective coating to tissues. Such a protective coating can prevent an undesired adhesion of the tissues. Two of the four lumina can then serve for supplying a two-component substance. A further lumen can serve for supplying a turbulence gas.

The multi-lumen tube may also have more than four lumina.

Exemplary embodiments of the invention shall be explained in greater detail below based on the drawings. Therein:

### BRIEF DESCRIPTION OF THE DRAWINGS

FIG. 1 shows, schematically, a tube system, designed as a multi-lumen tube system for medical applications, viewed from the side;

FIG. 2 shows, in a longitudinal view, a guide wire-tube lumen of a multi-lumen tube of the multi-lumen tube system according to FIG. 1;

FIG. 3 shows a section through the multi-lumen tube along line III-III in FIG. 1; and

FIGS. 4 to 6 show, respectively, a depiction similar to that in FIG. 3, of further designs for the multi-lumen tubes for use in a multi-lumen tube system according to the type in FIGS. 1 and 2.

### DETAILED DESCRIPTION OF THE PREFERRED EMBODIMENTS

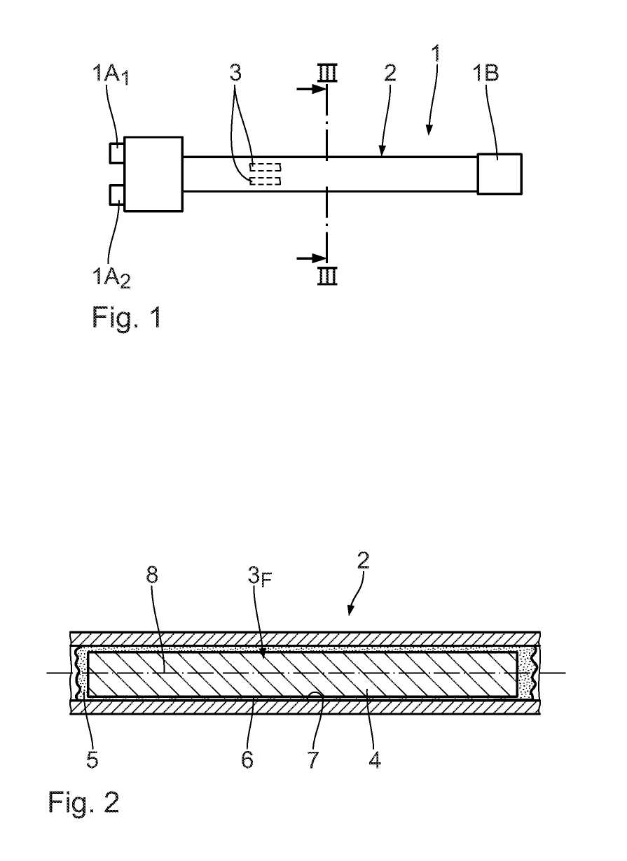

A tube system 1 that is schematically depicted in FIG. 1 is used, for example, in the processing of multi-component fibrin glues in medical interventions.

The tube system 1 has a tube 2 with numerous tube lumina 3, two of which tube lumina 3 are indicated by a broken line in FIG. 1. The tube 2 shall also be referred to below as a multi-lumen tube. The tube lumina 3 can have an inner diameter in the range of 0.5 to 5 mm.

The tube 2 is made of plastic. The tube 2 can be made, in particular, of polyvinylchloride (PVC) or silicone. The tube 2 can also be made of a thermoplastic.

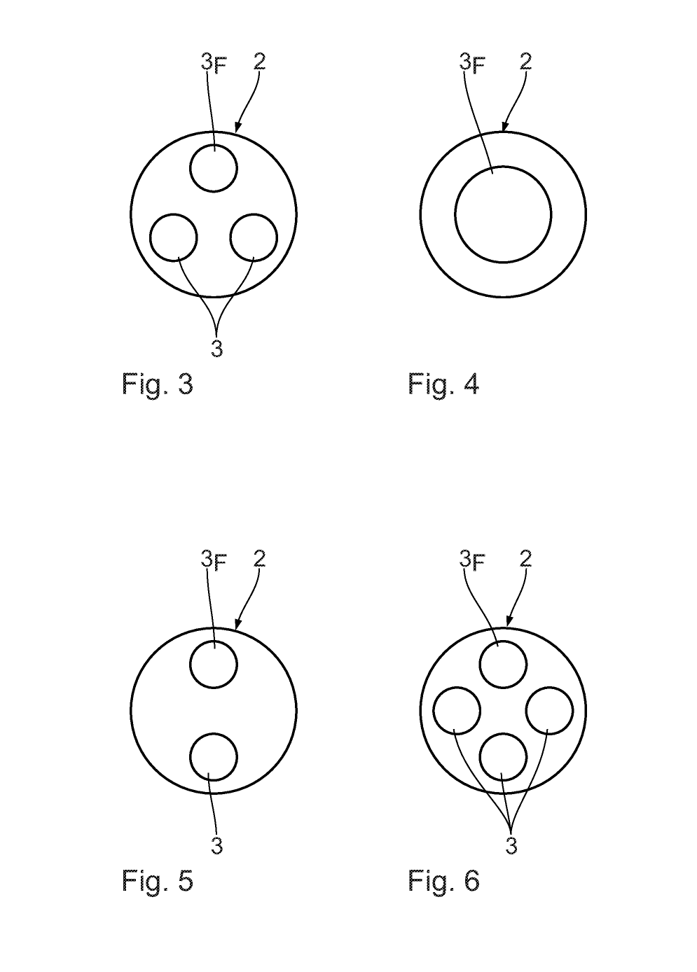

FIG. 3 shows a cross section through the tube 2. The tube 2 has a total of three tube lumina 3, one of which is used as the guide wire-tube lumen, and which shall also receive the reference symbol 3_{F} below.

FIG. 2 shows a longitudinal section of a section of the tube 2 in the region of the guide wire-tube lumen 3_{F}. A guide wire 4, also referred to as a mandrin, is inserted in a tube section in the guide wire-tube lumen 3_{F}. An adhesive coating 5 is disposed between an outer wall 6 of the inserted guide wire and an inner wall 7 of the guide wire-tube lumen 3_{F}.

The adhesive coating 5 provides for a fixing of the guide wire 4 in the guide wire-tube lumen 3_{F} and thus for a securing the guide wire 4 against torsion in the tube 2. The material of the tube 2 is compatible with the material of the adhesive coating 5, such that the tube 2 can be glued to the adhesive coating 5. A corresponding compatibility also applies to the material of the guide wire 4.

The guide wire 4 can be deformed in a plastic manner. The guide wire 4 is implemented as a silver-plated copper wire. The guide wire 4 provides for a stiffening of the tube 2 and thus for an improvement in the guidance. A length of the guide wire 4 lies in the range of 50 mm to 200 mm. It is also possible to use a guide wire here having a length of up to 600 mm, or even longer. Because of the securing against torsion, the tube 2 cannot yield to a torsional force applied to it by an operator, in particular, by means of a relative torsion of the guide wire-tube lumen 3_{F} in relation to the guide wire 4 with respect to a longitudinal axis 8 of the guide wire-tube lumen 3_{F}.

The adhesive coating 5 entirely encases the guide wire 4, thus also in the region of its end surfaces. As a result, a tight sealing of the guide wire 4 toward the exterior and thus a more efficient protection against corrosion of the guide wire 4 is obtained.

The adhesive coating 5 is formed by a controlled hardening adhesive. With the exemplary embodiment described here, the adhesive of the adhesive coating 5 is a UV-curing adhesive having a cyanoacrylate base. Another adhesive that can be cured in a targeted manner, and for example, hardens under the effects of humidity and/or temperature, or an adhesive having a longer curing time, can be used for the adhesive coating 5.

The tube system 1 with the tube 2 can be used as a dosing system for a two-component fibrin glue. The two components of the two-component fibrin glue are supplied to a deployment location via the two tube lumina 3 in which the guide wire 4 has not been inserted. Depending on the number of tube lumina 3, which can also be greater than two, a corresponding number of media can be guided through the tube 2.

The tube system 1 and in particular the tube 2 having the guide wire 4 are produced in the following manner: first, the guide wire 4 and the tube 2 are cut to a fixed length. The tube 2 is cut with an excess length thereby. Subsequently, the guide wire 4 is inserted in the guide wire-tube lumen 3_{F} and fixed in a desired axial position in the guide wire-tube lumen 3_{F}. Subsequently, the guide wire-tube lumen 3_{F} is completely filled in the region of the guide wire 4 with the adhesive, which at this point in time is a low-viscosity fluid. This is achieved by exploiting the capillary effect. Alternatively, or additionally, a vacuum may be created in this lumen in order to promote a filling of the guide wire-tube lumen 3_{F} in the region of the guide wire 4 with the adhesive. The low-viscosity adhesive is transported axially thereby from an adhesive application end, through the entire intermediate space between the outer wall 6 of the inserted guide wire 4 and the inner wall 7 of the guide wire-tube lumen 3_{F}, until reaching the end surface of the guide wire 4 lying opposite the adhesive application end. Adhesive coatings are formed thereby that cover the end surfaces of the guide wire 4, such that the guide wire 4 is entirely encased in adhesive. Subsequently, the adhesive is cured to form the adhesive coating 5. The ends of the guide wire-tube lumen 3_{F} that are not encased can then be cut off.

Aside from the tube 2, a media supply connection 1A having two supply ports 1A₁ and 1A₂ is comprised in the tube system 1 according to FIG. 1. Furthermore, a mixing component 1B for mixing media supplied via the tube lumina 3 is also comprised in the tube system 1. The mixing component 1B can be designed as a nozzle. Various media can be conveyed via the tube lumina 3; one and the same medium, however, can also be conveyed via the tube lumina 3.

FIGS. 4 to 6 show further variations of the tubes 2, which can be used in tube systems 1 instead of the multi-lumina tube according to FIG. 3.

The tube 2 according to FIG. 4 has a single tube lumen 3, which simultaneously represents the guide wire-tube lumen 3_{F}. When using a single lumen tube according to FIG. 4, a supply connection according to the type of connection 1A and a mixing component according to the type of mixing component 1B are no longer needed for the tube system.

The tube 2 according to FIG. 5 has two tube lumen 3, one of which represents the guide wire-tube lumen 3_{F}. A medium can be supplied or discharged via the other tube lumen 3.

The tube 2 according to FIG. 6 is designed as a four-lumina tube, wherein one of these four tube lumina 3 depicts, in turn, the guide wire-tube lumen 3_{F}. Two of the three tube lumina 3 of the four-lumen tube 2 that are not used as the guide wire-tube lumen 3_{F} can be used, in turn, to transport a two-component material. The remaining fourth tube lumen 3 can then be used to transport a turbulence gas. The four-lumen tube 2 used in this manner may be used in a dosing system for applying a protective coating to a tissue, wherein a protective coating of this type prevents an undesired adhesion of tissues.

1. A tube system for medical applications comprising:
a tube with at least one tube lumen,
a guide wire that is inserted at least in a tube section of the at least one tube lumen,
an adhesive coating between an outer wall of the inserted guide wire and an inner wall of the at least one tube lumen, and
wherein the adhesive coating is implemented such that it provides for a securing against torsion of the guide wire in relation to the tube.

2. The tube system according to claim 1, wherein the adhesive coating and the guide wire are substantially fully encased.

3. The tube system according to claim 1, wherein the guide wire is comprised of a copper wire.

4. The tube system according to claim 1, wherein the adhesive coating is formed by a controlled hardening adhesive.

5. The tube system according to claim 1, wherein the tube is comprised of a multi-lumen tube.

6. The tube system according to claim 5, wherein the multi-lumen tube is comprised of a three-lumen tube.

7. The tube system according to claim 5, wherein the multi-lumen tube is comprised of a four-lumen tube.

8. The tube system according to claim 4, wherein the controlled hardening adhesive is a UV-curing adhesive.

9. The tube system according to claim 4, wherein the controlled hardening adhesive is comprised of a cyanoacrylate base.

10. The tube system according to claim 3, wherein the guide wire comprises a silver-plated copper wire.

11. The tube system according to claim 1, wherein the adhesive fixes the guide wire to the at least one tube lumen.

12. A tube system for medical applications comprising:
an elongate tube having at least a plurality of tube lumens with at least one of the tube lumens comprising a guide-wire tube lumen;
a guide wire received in the guide wire tube lumen; and
an adhesive received in the guide wire tube lumen disposed between the guide wire and the guide-wire tube lumen fixing the guide wire in the guide-wire tube lumen and imparting increased torsional resistance to the tube.

13. The tube system according to claim 12, wherein the adhesive comprises a cyanoacrylate adhesive.

14. The tube system according to claim 12, wherein the adhesive comprises a controlled hardening adhesive.

15. The tube system according to claim 14, wherein the adhesive comprises a UV-cured adhesive.

16. The tube system according to claim 15, wherein the adhesive has a cyanoacrylate base.

17. The tube system according to claim 14, wherein the tube comprises at least three lumens.

18. The tube system according to claim 12, wherein the adhesive comprises a controlled hardening adhesive.

19. The tube system according to claim 12, wherein the adhesive comprises a UV-cured adhesive.

20. The tube system according to claim 12, wherein the adhesive comprises a cyanoacrylate adhesive.

21. The tube system according to claim 12, wherein the adhesive substantially completely encases the guide wire in the guide-wire tube lumen.
